(19) United States
(12) Patent Application Publication
(10) Pub. No.: US 2003/0060420 A1
(43) Pub. Date: Mar. 27, 2003

(54) Process for extraction and stabilization of phytoestrogens from flaxseed and product therefrom

(76) Inventors: Rick Heintzman, Onaka, SD (US); Michael L. Bennett, Vista, CA (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 10/041,327

(22) Filed: Jan. 8, 2002

Related U.S. Application Data
(60) Provisional application No. 60/322,253, filed on Sep. 14, 2001.

(51) Int. Cl. A61K 31/70; A61K 31/385; A61K 31/355; A61K 35/78

(52) U.S. Cl. 514/22; 514/25; 514/458; 514/440

(57) ABSTRACT

The phytoestrogen secoisolariciresinol diglucoside (SDG) and other beneficial compounds are extracted from oil-free flaxseed meal or flaxseed hulls. The phytoestrogens are extracted by aqueous ethanol solvent, which is then filtered and removed by evaporation to isolate the complexed phytoestrogens. The complexed phytoestrogens are then hydrolyzed with a base to provide uncomplexed phytoestrogens. The uncomplexed phytoestrogens are subsequently freeze dried and stabilized with a vegetable oil. The stabilized phytoestrogens can be formulated into products for consumption.

### CROSS-REFERENCES TO RELATED APPLICATIONS

This application claims priority under 35 U.S.C. §119(e) to, and incorporates by reference U.S. Provisional Patent Application No. 60/322,253, filed Sep. 14, 2001.

### BACKGROUND OF THE INVENTION

1. Field of the Invention

This invention relates to extraction and stabilization of phytoestrogens from plant materials and, in particular, this invention relates to extraction and stabilization of phytoestrogens, such as lignans, from flaxseed.

2. Background of the Invention

Phytoestrogens are naturally occurring chemicals derived from plants. These compounds are similar in structure to mammalian estrogens and have similar effects on mammals Several advantageous medicinal properties of phytoestrogens have been reported. One class of phytoestrogens is lignans. In flaxseed lignans constitute the majority of phytoestrogens present. Lignans have structural similarities to human steroidal estrogens. In mammals, lignans may have roles with agonist (activating estrogen receptors) or antagonist (blocking estrogen receptors) functions. Moreover, these compounds may themselves selectively bind to classical estrogen receptors in certain target tissues. In addition to, or possibly as a result of, estrogen receptor functions, lignans may be of value in preventing or treating cancer, atherosclerosis, lupus nephritis, and diabetes. One reported characteristic of lignans, which may be at least partially responsible for some of these medicinal advantages, is the utility of lignans as antioxidants.

The first known report of a new phytoestrogenic complex (termed a “new diglucoside”) was published by Bakke and Klosterman (Proceedings of the North Dakota Academy of Science 10: 18-22 (1956)), wherein methanol and barium methoxide were used to hydrolyze this polymer into a series of less complex compounds. One of the substances identified was secoisolariciresinol diglucoside (SDG).

The first known interest in SDG mammalian activity came in the late 1970s, when the presence of diphenolic compounds was reported in urine samples of females showing menstrual cycle variation. Other papers contained accounts of higher levels of these substances in urine samples of tumor-free females than in females with breast cancer. See, Aldercreutz et al., “Excretion of the lignans enterolactone and enterodiol and of equol in omnivorous and vegetarian postmenopausal women and in women with breast cancer,” Lancet: 1295-1299 (1982).

Several studies have shown substantial benefits when lignans are consumed in diets. See, e.g., Thompson, “Anticarcinogenic effect of a mammalian lignan precursor from flaxseed,” Proceedings 55th Flax Institute of U.S.A., Fargo, N. Dak., 46-50 (1994); U.S. Pat. No. 5,827,256 to Clark; U.S. Pat. No. 5,846,944 to Prasad. Lignans are present in extremely small concentrations in almost all food sources. Therefore, consuming enough lignans to obtain these benefits from most food sources is difficult or impossible. By contrast, flaxseed represents one exception to the foregoing fact. However, heretofore extracting pure stable quantities of lignans sufficient for nutritional supplements and medicinal purposes has not been possible.

One report (Harris et al., Midwest Research Inst. Final Report, December 1991) detailed using methanol/dioxane, methanol alone and ethanol/dioxane and ethanol alone to extract and analyze lignans from flaxseed. The ethanol/dioxane combination produced the highest yield of lignans (3.2 mcg SDG/g raw flaxseed). However, dioxane is extremely toxic. Therefore, using dioxane as a solvent is extremely problematic from the standpoint of safety and residual solvent elimination.

U.S. Pat. No. 5,705,618 describes a process for extracting lignans from flaxseed. However, this process requires expensive equipment and lengthy purification steps. Moreover, several phytoestrogens extracted by the disclosure of U.S. Pat. No. 5,705,618 may be either degraded or eliminated. Furthermore, no methods are described to stabilize these hygroscopic, hence unstable, lignans once they are isolated and purified.

Even though lignan levels in flax are relatively high, when compared to other plant sources, lignans are nonetheless present in low concentrations (1%-2%). Since the concentrations of lignans in flax are relatively low, commercial production is less feasible if lengthy and expensive process steps and/or equipment is required. Moreover, the amount of solvents required in the initial steps are usually quite large. Consequently, large amounts of time, solvents and equipment are often required to obtain relatively small amounts of the desired product. Moreover, residual amounts of the solvents used may remain. Hence, the use of any solvent other than ethanol and water presents additional problems of ensuring that residual solvents do not appear in the final product.

It is known in herbal medicine that the most effective extracts are often those having several compounds, rather than a single compound. In contrast to this fact, U.S. Pat. No. 5,705,618 discloses using anion exchange and reverse phase resins to effectively “strip” away several potentially important compounds such as other phytoestrogens. These potentially beneficial compounds include cinnamic acid glucosides, amino acids, peptides, and hydroxy methyl glutaric acid.

Another concern not addressed by the disclosure of U.S. Pat. No. 5,705,618 is stabilization of lignans after extraction. Dried lignans are very hygroscopic and form a sticky resinous, gum-like material after a brief exposure to atmospheric humidity. When left exposed, this hydrated material supports the growth of molds and bacteria. As this material absorbs moisture, it becomes extremely difficult to handle and formulate into capsules and loses quality due to the effects of molds and bacteria.

U.S. Pat. No. 6,264,853 B1 discloses isolating a complex containing lignans and phenolic and aliphatic substances from flax and a process for preparing these substances. However, the lignans are isolated and purified in a complexed (nonhydrolyzed) form. These lignans are also not stabilized so that they do not absorb ambient moisture. Therefore, the lignans isolated by this protocol are difficult to use in formulations after even a small amount of time after being extracted. Moreover, several potentially beneficial compounds are excluded by the very selective ultrafiltration. Additionally, the materials and other apparatus required may be too expensive to economically extract commercial quantities of lignans and related compounds.

There is then a need for a process for economically extracting commercially viable quantities of uncomplexed phytoestrogen, i.e. lignans which have been stabilized against absorbing moisture and from microbial contamination. There is a particular need for a process for economically extracting commercially viable quantities of SDG and other lignan-related components.

### SUMMARY OF THE INVENTION

This invention substantially meets the aforementioned needs of the industry by providing a process for economically extracting and stabilizing commercially viable quantities of uncomplexed SDG and other lignan-related components.

In one embodiment, lignans are extracted from oil-free flaxseed meal and stabilized by adding vegetable oils. The lignans are extracted from the oil-free flaxseed meal by adding aqueous ethanol and agitating the flaxseed meal and aqueous ethanol. The aqueous ethanol and dissolved lignans are separated from the flaxseed meal and the aqueous ethanol is then filtered to remove particulates. The aqueous alcohol solution is then evaporated to provide concentrated, complexed lignans and other compounds, such as glucosides, amino acids, small peptides, sugars, and salts. In addition to the lignan SDG, other lignans present may include cinnamic acid glucosides and hydroxy methyl glutaric acid. The lignans are hydrolyzed by an alkaline metal hydroxide, then neutralized with an acid to provide uncomplexed lignans. The uncomplexed lignans are dried, then stabilized by adding vegetable oils. The vegetable oils prevent the otherwise hygroscopic lignans from absorbing moisture from ambient air. The vegetable oils are stabilized, hence the lignans are further stabilized, by adding exogenous antioxidants such as tocopherols and/or tocotrienols. The stabilized lignans may be consumed directly or formulated as soft gel caps, hard capsules, or as a functional beverage.

It is one feature of the present invention to extract phytoestrogens, such as lignans, from a vegetable material, such as flaxseed, using relatively inexpensive equipment and materials and few processing steps.

It is another feature of the present invention to provide a process for extracting phytoestrogens, such as lignans, from a vegetable material, such as flaxseed, the process greatly reducing or eliminating residual amounts of toxic solvents present in the final product.

It is yet another feature of the present invention to provide a process for extracting phytoestrogens, such as lignans, from a plant material, such as flaxseed, to obtain a product in which other potentially beneficial compounds are present.

It is still another feature of the present invention to provide a process for extracting phytoestrogens, such as lignans, from a vegetable material, such as flaxseed, to produce a “broad spectrum” of beneficial compounds with potentially greater bioactivity.

It is yet still another feature of the present invention to provide a process for extracting and stabilizing phytoestrogens, such as lignans, from plant materials, such as flaxseed,.

It is an additional feature of the present invention to provide a process for extracting and stabilizing phytoestrogens, such as lignans, from plant materials, such as flaxseed, which are not conducive to bacterial and fungal growth and development.

It is a yet additional feature of the present invention to provide a process for extracting and stabilizing phytoestrogens, such as lignans, from plant materials, such as flaxseed, which can be easily and inexpensively formulated for consumption.

Additional objects, advantages, and features of various embodiments of the invention will be set forth in part in the description which follows, in part will become apparent to those skilled in the art upon examination of the following, or may be learned by practice of the invention.

### BRIEF DESCRIPTION OF THE FIGURES

FIG. 1 is a flowchart of one embodiment of the present process for extracting and stabilizing lignans from plant materials;

FIG. 2 is a chromatogram of a phytoestrogen polymer after ethanol/water extraction and before hydrolysis;

FIG. 3 is a chromatogram of the phytoestrogen components, including secoisolariciresinol diglucoside (SDG), after ethanol/water extraction and hydrolysis; and

FIG. 4 is a chromatogram of SDG, a major phytoestrogen component of the final product extracted by the present process.

It is understood that the above-described figures are only illustrative of the present invention and are not contemplated to limit the scope thereof.

### DETAILED DESCRIPTION OF THE INVENTION

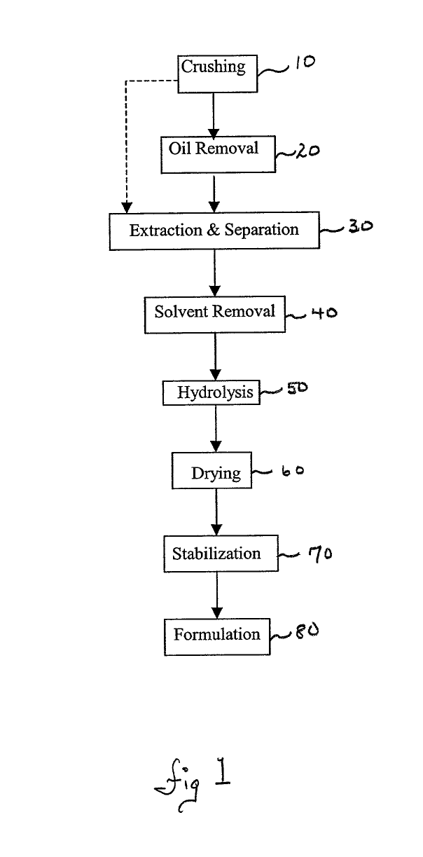

One embodiment of the process of this invention is a protocol for extracting and stabilizing phytoestrogens, such as phytoestrogens, from plant materials, such as flaxseed. This protocol is illustrated in FIG. 1 and is discussed more fully below. While the present protocol is contemplated to include recovery of phytoestrogens from plant material of any species, one suitable taxon is the family Linaceae. A suitable species within the family Linaceae is flax (Linium usitatissimum L.). The phytoestrogens extracted and stabilized by the present protocol primarily include lignans, such as secoisolariciresinol diglucoside (SDG). However, other compounds, such as cinnamic acid glucosides and hydroxy methyl glutaric acid, may also be present. Still other beneficial substances, which may also be present in the extract from the present protocol, include amino acids, small peptides (e.g., less than 30 residues), simple sugars, and undefined salts. While whole flaxseed can be utilized in the present extraction process, oil-free flaxseed meal is usually the raw material of choice. Whole flaxseed contains vegetable oils which are valuable per se. Suitable results have been obtained when the flaxseed is crushed and the vegetable oils are subsequently extracted from the crushed flaxseed before phytoestrogens are extracted (as indicated in FIG. 1 at 10, 20, and 30). Oil-free flaxseed meal is considered to be the meal from which flaxseed oil has been extracted by any process known to the art. While the oil-free flaxseed meal may not be totally free from indigenous vegetable oils, the indigenous vegetable oils will be present in very low amounts, e.g., less than about 0.5% by weight. It is also recognized that the hulls of may oil seeds (e.g., flax) contain most of the lignans. Thus, the flaxseed mean should include the hulls or seed coats. Alternatively, the hulls per se could be used in the present extraction process in the absence of the remainder of the seeds.

At step 30, phytoestrogens are extracted and separated from the oil-free flaxseed meal by an aqueous aliphatic alcohol. Aliphatic alcohols suitable for extracting the phytoestrogens in the present process include methanol, ethanol, propanol, isopropanol, and butanol. In one embodiment, aqueous ethanol is used. While these alcohols may be used in substantially pure form, mixtures thereof with water may be used in some embodiments. Some suitable alcohol proportions in aqueous mixtures (v/v) include at least about 50 percent, between about 50 percent and 85 percent, between about 65 percent and 75 percent, and between about 60 percent, and 70 percent.

The aqueous alcohol may be added to the flaxseed meal in a liquid (volume) to solids (weight) ratio of from between about 2 and 50 liters aqueous alcohol per kilogram oil free flaxseed meal or between about 4 and 30 liters aqueous alcohol per kilogram flaxseed meal. The time in which the flaxseed meal is extracted in the aqueous alcohol may be between about 12 hours and 36 hours or between about 12 hours and 24 hours at ambient temperatures. However, under higher temperatures, the time in which the flaxseed meal is extracted may be between about one hour and six hours or between about one hour and four hours. As stated above, extraction may be performed at ambient temperatures. Temperatures between about 10 degrees Celsius and 60 degrees Celsius may be suitable under certain circumstances. Other temperatures, which may be suitable in some embodiments, are between about 10 degrees Celsius and 45 degrees Celsius and between about 20 degrees Celsius and 35 degrees Celsius.

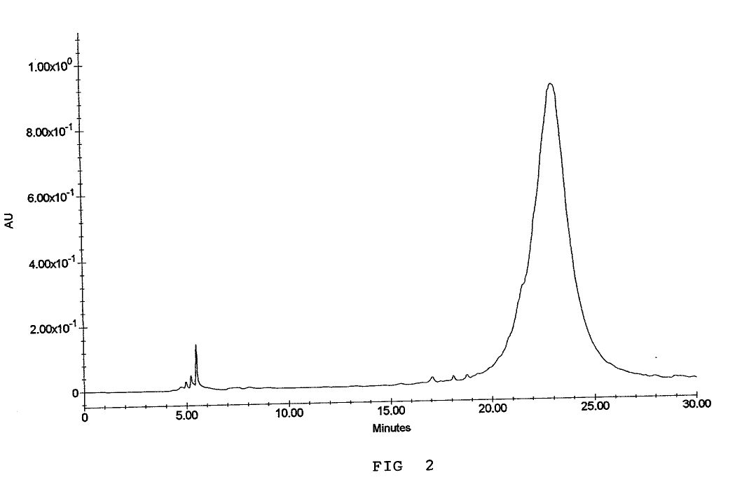

After the desired extraction time, the aqueous alcohol has dissolved complexed phytoestrogens from the flaxseed meal and can be separated from the flaxseed meal, e.g., by decanting, or other means known to the art. The remaining particulates are removed from the aqueous alcohol with the dissolved complexed phytoestrogens by, e.g., a series of filters. Filtering may be a multistage process, e.g., with a first stage removing particulates greater than about 250 microns and a second stage removing particulates greater than about 25 microns. One suitable series of filters is designated as Polyloc™, and is available from Filter Specialists, Inc., Michigan City, Ind. A final extracted aqueous alcohol solution with particulates greater than about 25 microns removed has been found to be suitable. After removal of particulates, a colored, yellow/orange solution results. FIG. 2 is a chromatogram of phytoestrogen polymers present after the alcohol/water extraction.

In step 60, the colored solution is heated, e.g., between about 50 degrees Celsius and 70 degrees Celsius, between about 55 degrees Celsius and 65 degrees Celsius or at about 60 degrees Celsius, under a partial vacuum of between about 500 and 600 mm Hg until the alcohol has been evaporated, thereby leaving a lignan-containing aqueous fraction.

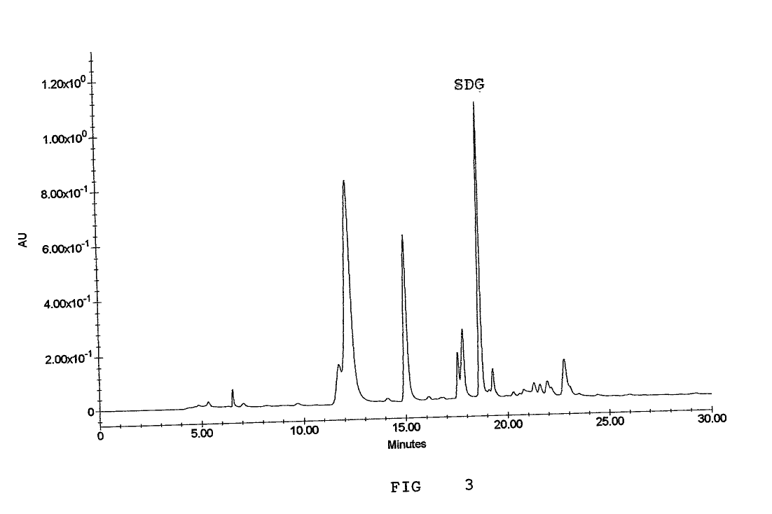

As indicated by step 50, the aqueous lignan fraction may then be hydrolyzed with a base to generate uncomplexed lignans. The base may be an alkali metal base, such as sodium hydroxide or potassium hydroxide. Hydrolysis may be allowed to proceed for between about 1 and 2 hours, at a pH between about 12 and 13, and at a temperature between about 40 degrees Celsius and 70 degrees Celsius, between about 50 degrees Celsius and 70 degrees Celsius or at about 60 degrees Celsius. After hydrolysis is complete, sufficient acid (e.g., hydrochloric acid) is added to adjust the pH to between 7 and 8. However, a person of ordinary skill in the art will be able to readily determine other temperatures, pH levels, and durations at which hydrolysis may be conducted. A yellow-brown solution may result after the pH has been adjusted to between about 7 and 8. FIG. 3 is a chromatograph of the phytoestrogenic components present after hydrolysis.

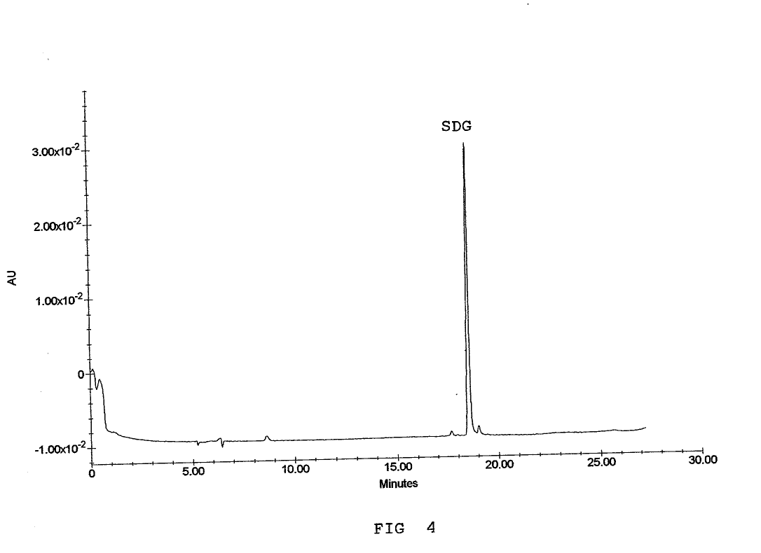

The yellow-brown solution obtained hereinabove may then be dried in step 60, e.g., by a freeze-drying, to obtain anhydrous yellow crystals. FIG. 4 is a chromatograph of SDG present in the crystals produced by the present protocol through step 60.

The crystals resulting from the foregoing drying protocol are extremely hygroscopic in nature, hence readily absorb moisture from the atmosphere. Because of this hygroscopic property, the powder with the uncomplexed phytoestrogens cannot be stored, even for short periods of time without absorbing sufficient water to render it unstable, unsuitable, or difficult for use in making edible formulations. To avoid absorbing water, the crystals are subsequently stabilized by being suspended in a stabilizer, such as one or more vegetable oils and as indicated at 70. Vegetable oils suitable to be used as stabilizers include virtually any edible oil of vegetable origin. A nonlimiting example of stabilizers includes oils from jojoba, apricot, sesame, grapeseed, olive, sunflower, corn, canola, soy, peanut, and palm. Alternatively, high oleic vegetable oils may be desirable under certain circumstances. High oleic vegetable oils are considered to be vegetable oils with oleic acid proportions greater than about 60 percent. Examples of high oleic vegetable oils include oils from corn, sunflower, canola, peanut, and olive. Between about 15% and 30%, or between about 20% and 25% vegetable oils may be added to the powder by weight.

Exogenous antioxidants, such as alpha lipoic acid, tocopherols, tocotrienols, and carotenoids, may also be added. These exogenous antioxidants serve to prevent degradation (oxidation) of the oil. Therefore, these exogenous antioxidants themselves further serve to stabilize the phytoestrogens extracted by the present process.

The stabilized extracted crystals may be consumed directly or may be prepared in formulations such as soft gel caps or hard capsules. Soft gel caps may be made by coating formulations of the present crystals and allowing the coating to harden (e.g., polymerize). In some instances, the stabilized crystals are fine milled in the vegetable oil before encapsulation.

In formulating the stabilized crystals for hard capsules, the present crystals may be fine milled in the vegetable oil. Alternatively the present crystals may be ground to a size range between about 10 and 30 mesh, between about 15 and 25 mesh, or about 20 mesh before being encapsulated in hard (e.g., gelatin) capsules. Between about 200 mg and 400 mg or about 300 mg of crystals may be present in some embodiments of hard or soft capsules.

### EXAMPLE I

Oil-free flaxseed meal was obtained from a commercial crusher. A 10 kg quantity of flaxseed meal was continuously mixed at ambient temperature (about 20 degrees Celsius) with 80 liters of 70% (v/v) aqueous ethanol in a batch container for 24 hours. The resulting suspension was filtered in a series of filters so as to remove particulates greater than 25 microns. The flaxseed meal fines were then discarded. The colored solution was transferred to a simple pot still and heated to 60 degrees Celsius under a vacuum of 500 mm Hg until the alcohol had been evaporated away, thereby leaving an aqueous fraction.

The aqueous fraction was hydrolyzed for two hours, at 60 degrees Celsius, with sufficient sodium hydroxide to maintain a pH of between 12 and 13. The hydrolysis was stopped by adding sufficient HCl to adjust the pH to between 7 and 8, thereby generating a yellow-brown solution. The yellow-brown solution was then concentrated and freeze-dried to produce a yellow lignan powder. Thirty grams of the yellow powder was stabilized by being suspended in 16 g of sesame oil.

### EXAMPLE II

Forty-six g of the stabilized yellow lignan powder from Example I was further stabilized by adding 1.5 g of vitamin E. The lignan suspension was then fine milled to reduce particle size of the phytoestrogens therein.

### EXAMPLE III

A two-piece hard gelatin capsule was made using the lignan suspension from Example II. The formulation included:

| Ingredient | Proportion |
|---|---|
| Lignan crystals | 50% |
| Sesame oil | 27% |
| Vitamin E | 2% |
| MBK fatty acid base | 20% |
| Lipoic acid | 1% |

### EXAMPLE IV

A soft gelatin capsule was made using the lignan suspension from Example II. The formulation included:

| Ingredient | Proportion |
|---|---|
| Lignan crystals | 70% |
| Sesame oil | 27% |
| Vitamin E | 2% |
| Lipoic acid | 1% |

### EXAMPLE V

Oil is extracted from 100 pounds of crushed flaxseed to yield 60 pounds of oil-free flaxseed meal. Six pounds of lignans are then extracted from the oil-free flaxseed meal by the protocol of Example I for an extracted-lignan yield of 6%.

### EXAMPLE VI

One hundred pounds of flaxseed is dehulled to provide 12 pounds of flaxseed hulls. By using the protocol of Example I, six pounds of lignans are then extracted from the flaxseed hulls. As can be seen from Examples V and VI, essentially all of the extractable lignans are present in the hulls of flaxseed.

Because numerous modifications of this invention may be made without departing from the spirit thereof, the scope of the invention is not to be limited to the embodiments illustrated and described. Rather, the scope of the invention is to be determined by the appended claims and their equivalents.

What is claimed is:

1. A formulation, comprising:
a stabilizer; and
an uncomplexed phytoestrogen mixed with the stabilizer

2. The formulation of claim 1, the phytoestrogen comprising a lignan.

3. The formulation of claim 2, the lignan comprising secoisolariciresinol diglucoside.

4. The formulation of claim 3, in which the lignan is emulsified in the stabilizer.

5. The formulation of claim 3, the stabilizer comprising a vegetable oil.

6. The formulation of claim 3, the stabilizer comprising a vegetable oil selected from jojoba oil, apricot oil, sesame oil, grape seed oil, olive oil, sunflower oil, corn oil, canola oil, soy oil, peanut oil, and palm oil.

7. The formulation of claim 3, further comprising an exogenous antioxidant.

8. The formulation of claim 7, the exogenous antioxidant comprising a tocopherols or a tocotrienol.

9. The formulation of claim 7, the exogenous antioxidant comprising alpha lipoic acid.

10. The formulation of claim 3, in which the lignan is from a seed, the seed from the family Linaceae.

11. The formulation of claim 3, in which the lignan is from a flaxseed.

12. A process of obtaining stabilized phytoestrogens from a plant material containing complexed phytoestrogens, comprising:
extracting the complexed phytoestrogens from the plant material with an aqueous aliphatic alcohol;
separating the aqueous aliphatic alcohol and extracted complexed phytoestrogens from the plant material;
evaporating the aqueous aliphatic alcohol, thereby leaving a resinous material, the resinous material comprising the extracted complexed phytoestrogens;
hydrolyzing the resinous material with a base, thereby obtaining uncomplexed phytoestrogens;
neutralizing the uncomplexed phytoestrogens with an acid;
drying the uncomplexed phytoestrogens; and
stabilizing the uncomplexed phytoestrogens.

13. The process of claim 12, in which the extracted phytoestrogens comprise lignans.

14. The process of claim 12, in which the extracted phytoestrogens comprise secoisolariciresinol diglucoside.

15. The process of claim 14, in which extracting complexed phytoestrogens comprises agitating the aqueous aliphatic alcohol and the plant material.

16. The process of claim 14, in which the complexed phytoestrogens are extracted with aqueous ethanol.

17. The process of claim 14, in which the complexed phytoestrogens are extracted with aqueous ethanol, said aqueous ethanol including ethanol present in a proportion between about 50 percent and 85 percent, by volume.

18. The process of claim 14, in which the complexed phytoestrogens are extracted at a temperature less than 60 degrees Celsius.

19. The process of claim 14, in which the complexed phytoestrogens are extracted at a temperature between about 20 degrees Celsius and 35 degrees Celsius.

20. The process of claim 14, in which the complexed phytoestrogens are extracted for a period of 12 hours to 36 hours.

21. The process of claim 14, in which the complexed phytoestrogens are extracted for a period of 12 hours to 24 hours.

22. The process of claim 14, in which the aqueous aliphatic alcohol and extracted complexed phytoestrogens are separated from the plant material with a series of filters.

23. The process of claim 14, in which the aqueous aliphatic alcohol and extracted complexed phytoestrogens are separated from the plant material with a series of filters configured to remove particulates greater than 25 microns in diameter.

24. The process of claim 14, in which the resinous material is hydrolyzed with an alkali metal hydroxide.

25. The process of claim 14, in which the resinous material is hydrolyzed with sodium hydroxide.

26. The process of claim 14, in which the uncomplexed phytoestrogens are dried by freeze drying.

27. The process of claim 14, in which the uncomplexed phytoestrogens are dried at a temperature less than about 60 degrees Celsius.

28. The process of claim 14, in which the uncomplexed phytoestrogens are stabilized by being emulsified in a stabilizer.

29. The process of claim 14, in which the uncomplexed phytoestrogens are stabilized by adding a vegetable oil.

30. The process of claim 29, in which the uncomplexed phytoestrogens are further stabilized by adding an exogenous antioxidant.

31. The process of claim 29, in which the uncomplexed phytoestrogens are further stabilized by adding an exogenous antioxidant, the added antioxidant comprising a tocopherol.

32. The process of claim 29, in which the uncomplexed phytoestrogens are further stabilized by adding alpha lipoic acid.

33. An edible formulation made by the process of claim 14.

34. An edible formulation made by the process of claim 29.

35. An edible formulation comprising:
a substantially anhydrous, uncomplexed lignan comprising secoisolariciresinol diglucoside;
a stabilizer; and
a capsule base.

36. The formulation of claim 35, the stabilizer comprising a vegetable oil.

37. The formulation of claim 35, the anhydrous uncomplexed lignan present in an amount between about 42 percent and 83 percent, by weight of the formulation.

38. The formulation of claim 36, further comprising an exogenous antioxidant, the exogenous antioxidant including a tocopherol.

39. The formulation of claim 36, the exogenous antioxidant further comprising lipoic acid.

40. A process of obtaining stabilized lignans from flaxseed hulls, comprising:
extracting complexed phytoestrogens from the flaxseed hulls with an aqueous aliphatic alcohol;
separating the aqueous aliphatic alcohol and extracted complexed lignans from the flaxseed hulls;
evaporating the aqueous aliphatic alcohol, thereby leaving a resinous material, the resinous material comprising the extracted complexed lignans;
hydrolyzing the resinous material with a base, thereby obtaining uncomplexed lignans;
neutralizing the uncomplexed lignans;
drying the uncomplexed lignans; and
stabilizing the uncomplexed lignans.

41. The process of claim 40, in which the extracted lignans comprise secoisolariciresinol diglucoside.

42. The process of claim 40, in which extracting complexed lignans comprises agitating the aqueous aliphatic alcohol and the flaxseed hulls.

43. The process of claim 40, in which the complexed lignans are extracted with aqueous ethanol.

44. The process of claim 40, in which the complexed lignans are extracted with aqueous ethanol, the aqueous ethanol including ethanol present in a proportion between about 50 percent and 85 percent by volume.

45. The process of claim 40, in which the complexed lignans are extracted at a temperature less than about 60 degrees Celsius.

46. The process of claim 40, in which the complexed lignans are extracted at a temperature between about 20 degrees Celsius and 35 degrees Celsius.

47. The process of claim 40, in which the complexed lignans are extracted for a period of about 12 hours to 36 hours.

48. The process of claim 40, in which the complexed lignans are extracted for a period of about 12 hours to 24 hours.

49. The process of claim 40, in which the aqueous aliphatic alcohol and extracted complexed lignans are separated from the flaxseed hulls with a series of filters.

50. The process of claim 40, in which the resinous material is hydrolyzed with an alkali metal hydroxide.

51. The process of claim 40, in which the resinous material is hydrolyzed with sodium hydroxide.

52. The process of claim 40, in which the uncomplexed lignans are dried by freeze drying.

53. The process of claim 40, in which the uncomplexed lignans are dried at a temperature less than about 60 degrees Celsius.

54. The process of claim 40, in which the uncomplexed lignans are stabilized by being emulsified in a stabilizer.

55. The process of claim 40, in which the uncomplexed lignans are stabilized by adding a vegetable oil.

56. The process of claim 55, in which the uncomplexed lignans are further stabilized by adding an exogenous antioxidant.

57. The process of claim 55, in which the uncomplexed lignans are further stabilized by adding an exogenous tocopherol.

58. The process of claim 55, in which the uncomplexed lignans are further stabilized by adding alpha lipoic acid.

59. An edible formulation made by the process of claim 40.

60. An edible formulation made by the process of claim 55.
